(19) United States
(12) Patent Application Publication
(10) Pub. No.: US 2015/0265214 A1
(43) Pub. Date: Sep. 24, 2015

(54) ADJUSTABLE SENSOR SUPPORT STRUCTURE FOR OPTIMIZING SKIN CONTACT

(71) Applicant: Samsung Electronics Co., Ltd., Gyeonggi-do (KR)

(72) Inventors: Margreet De Kok, Eindhoven (NL); Lindsay Brown, Mierlo (NL); Gert Van Heck, Eindhoven (NL); Sheldon George Phillips, Glendale, CA (US); Joseph J. Kopp, JR., Fairfield, CA (US)

(73) Assignee: Samsung Electronics Co., Ltd., Gyeonggi-do (KR)

(21) Appl. No.: 14/284,532

(22) Filed: May 22, 2014

Related U.S. Application Data
(60) Provisional application No. 61/969,766, filed on Mar. 24, 2014.

(51) Int. Cl. A61B 5/00; H05K 3/10; A61B 5/1455; A61B 5/0205; A61B 5/0408

(52) U.S. Cl. CPC A61B 5/6843; A61B 5/02055; A61B 5/0408; A61B 5/742; A61B 5/7475; A61B 5/0015; A61B 5/14552; H05K 3/10

(57) ABSTRACT

Exemplary embodiments provide an adjustable sensor support structure for optimal skin contact. Aspects of the exemplary embodiments include a sensor array comprising a plurality of sensor units arranged on a band such that the sensor array straddles or otherwise addresses a blood vessel when worn on a measurement site of a user; and a pressure exertion apparatus attached between the sensor units and the band that exerts outward pressure on the sensor units towards the measurement site causing the sensor units to maintain contact with skin of the user independent of motion activity of the band, thereby improving contact quality, wherein the pressure exertion apparatus comprises at least one of: a flexible bridge structure, a flexible foam structure, and a sensor trampoline structure.

### CROSS-REFERENCE TO RELATED APPLICATIONS

The present application claims the benefit of U.S. Provisional Application No. 61/969,766, filed on Mar. 24, 2014, which is incorporated herein by reference in its entirety.

### BACKGROUND

Physiological data may be obtained through various measurements such as photoplethysmograph (PPG), electrocardiogram (ECG), bioimpedance, and others which may be implemented in a portable device.

For example, heart rate may be measured by detecting the impedance changes caused by a pulse in blood flow within a local area of the body. Heart rate detection through measurement of the electrical properties of flowing blood may be achieved by measuring the potential created by current passed through the blood, artery, and surrounding tissue. Conventional wearable heart rate detectors utilizing an impedance sensing scheme are often worn at or near the chest using a chest band. In another conventional implementation, the heart rate detector may instead be placed along the underside of the forearm with four electrodes arranged in a line along the radial artery.

As another example, an electrocardiogram is a manifestation of electrical potentials produced through cardiac activity as observed at the body surface and may be used to interpret various aspects related to cardiac activity. An ECG reading is typically acquired using electrodes placed on a user's skin. A motion artifact is noise that results from motion of the electrode in relation to the user's skin (i.e. electrode movement causes deformations of the skin around the electrode site in turn causing a change in the electrical characteristics of the skin about the electrode) and may make a contribution to the ECG reading that is not related to cardiac activity, thus creating the potential for misinterpretation of the ECG reading.

Motion artifacts are an especially major technical challenge faced when making ECG measurements in a mobile or portable application, as well as with heart rate detection, as dealing with a high level of noise introduced by motion artifacts becomes an increased component of the overall measured signal.

Accordingly, what is required is a system and methods to provide secure contact between a physiological parameter sensor and the measurement site upon the user such that the contact, and therefore the quality, of measured signals is improved.

### BRIEF SUMMARY

Exemplary embodiments provide an adjustable sensor support structure for optimal skin contact. Aspects of the exemplary embodiments include a sensor array comprising a plurality of sensor units arranged on a band such that the sensor array straddles or otherwise addresses a blood vessel when worn on a measurement site of a user; and a pressure exertion apparatus attached between the sensor units and the band that exerts outward pressure on the sensor units towards the measurement site causing the sensor units to maintain contact with skin of the user independent of motion activity of the band, thereby improving contact quality, wherein the pressure exertion apparatus comprises at least one of: a flexible bridge structure, a flexible foam structure, and a sensor trampoline structure.

### BRIEF DESCRIPTION OF SEVERAL VIEWS OF THE DRAWINGS

The features and utilities described in the foregoing brief summary, as well as the following detailed description of certain embodiments of the present general inventive concept below, will be better understood when read in conjunction with the accompanying drawings of which:

FIG. 1 is a diagram illustrating an embodiment of a modular sensor platform.

FIG. 2 is an embodiment of the modular sensor platform of FIG. 1.

FIG. 3 is a diagram illustrating another embodiment of a modular sensor platform.

FIG. 4 is a block diagram illustrating one embodiment of the modular sensor platform, including a bandwidth sensor module in connection with components comprising the base computing unit and battery.

FIG. 5 is a cross-sectional illustration of the wrist with a band mounted sensor in contact for an embodiment used about the wrist.

FIG. 6 is a diagram illustrating another embodiment of a modular sensor platform with a self-aligning sensor array system in relation to use about the wrist.

FIG. 7 is a block diagram illustrating components of the modular sensor platform including example sensors and an optical electric unit self-aligning sensor array system in a further embodiment.

FIG. 8 is a diagram of a cross section of a wrist and an embodiment of an adjustable sensor support structure according to a further aspect of the exemplary embodiment.

FIG. 9 is a diagram is a diagram of a cross section of the band and an embodiment where the pressure exertion apparatus comprises a flexible bridge structure.

FIGS. 10A through 10F illustrate an exemplary process for assembling a flexible bridge structure.

FIGS. 11 and 12 are diagrams illustrating one embodiment of multiple flexible bridge structures connected edge-to-edge on the band in a serial configuration.

FIG. 13 is a diagram illustrating yet another embodiment of the multiple flexible bridge structures in which the flexible bridge structures are layered on top of each other to form a multiple bridge structure spring.

FIG. 14 is a diagram is a diagram of a cross section of the band showing an embodiment where the flexible foam structure comprises foam islands.

FIGS. 15A and 15B are diagrams of a cross section of the band showing an embodiment where the flexible foam structure comprises flexible cavity structures.

FIG. 16 is a diagram illustrating an embodiment where the pressure exertion apparatus of the adjustable sensor support structure comprises a sensor trampoline structure.

### DETAILED DESCRIPTION

Reference will now be made in detail to the embodiments of the present general inventive concept, examples of which are illustrated in the accompanying drawings, wherein like reference numerals refer to the like elements throughout. The embodiments are described below in order to explain the present general inventive concept while referring to the figures.

Advantages and features of the present invention and methods of accomplishing the same may be understood more readily by reference to the following detailed description of embodiments and the accompanying drawings. The present general inventive concept may, however, be embodied in many different forms and should not be construed as being limited to the embodiments set forth herein. Rather, these embodiments are provided so that this disclosure will be thorough and complete and will fully convey the concept of the general inventive concept to those skilled in the art, and the present general inventive concept will only be defined by the appended claims. In the drawings, the thickness of layers and regions are exaggerated for clarity.

Also, the phraseology and terminology used in this document are for the purpose of description and should not be regarded as limiting. The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

As should also be apparent to one of ordinary skill in the art, the systems shown in the figures are models of what actual systems might be like. Some of the modules and logical structures described are capable of being implemented in software executed by a microprocessor or a similar device, or of being implemented in hardware using a variety of components including, for example, application specific integrated circuits (“ASICs”). A term like “processor” may include or refer to both hardware and/or software. No specific meaning is implied or should be inferred simply due to the use of capitalization.

The term “component” or “module”, as used herein, means, but is not limited to, a software or hardware component, such as a field programmable gate array (FPGA) or an application specific integrated circuit (ASIC), which performs certain tasks. A component or module may advantageously be configured to reside in the addressable storage medium and configured to execute on one or more processors. Thus, a component or module may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. The functionality provided for the components and components or modules may be combined into fewer components and components or modules or further separated into additional components and components or modules.

Unless defined otherwise, all technical and scientific terms used herein have the same meaning as commonly understood by one of ordinary skill in the art to which this invention belongs. It is noted that the use of any and all examples, or exemplary terms provided herein is intended merely to better illuminate the invention and is not a limitation on the scope of the invention unless otherwise specified. Further, unless defined otherwise, all terms defined in generally used dictionaries may not be overly interpreted.

Embodiments of the invention relate to an adjustable sensor support structure, which includes sensor units and a pressure exertion apparatus attached between the sensor units and a band. The band with the pressure exertion apparatus is worn about a measurement site of a user, such as a wrist, and exerts outward pressure on the sensor units towards the measurement site to compensate for varying topology of the measurements site and to cause the sensor pads to maintain proper contact with the user's skin independent of motion activity of the band (and any other physiological measurement device attached to the band). According to exemplary embodiments, the pressure exertion apparatus may include one or more of flexible island structures, flexible foam structures, and/or flexible meshes.

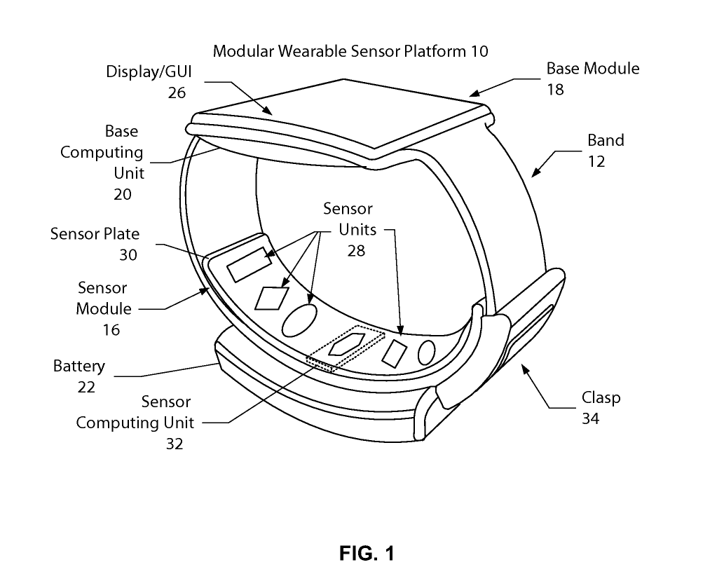

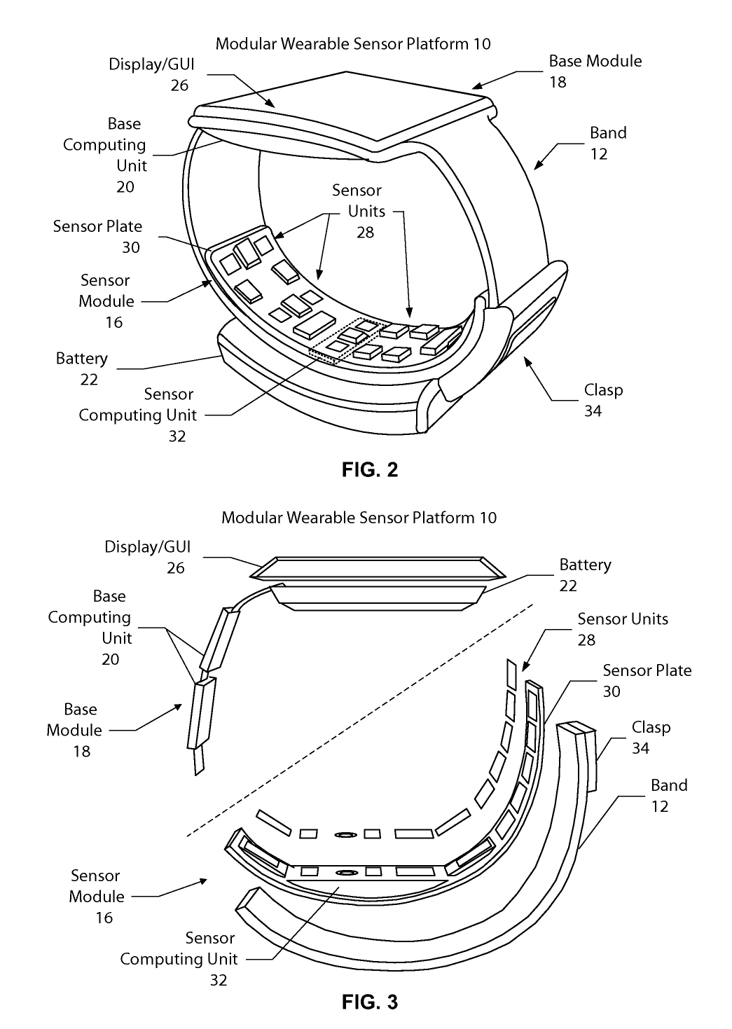

FIGS. 1 and 2 are diagrams illustrating embodiments of a modular wearable sensor platform. FIGS. 1 and 2 depict a perspective view of embodiments of the wearable sensor platform 10, while FIG. 3 depicts an exploded side view of another embodiment of the wearable sensor platform 10. Although the components of the wearable sensor platform in FIGS. 1 and 2 may be substantially the same, the locations of modules and/or components may differ.

In the embodiment shown in FIG. 1, the wearable sensor platform 10 may be implemented as a smart watch or other wearable device that fits on part of a body, here a user's wrist.

The wearable sensor platform 10 may include a base module 18, a band 12, a clasp 34, a battery 22 and a sensor module 16 coupled to the band 12. In some embodiments, the modules and/or components of the wearable sensor platform 10 may be removable by an end user (e.g., a consumer, a patient, a doctor, etc.). However, in other embodiments, the modules and/or components of the wearable sensor platform 10 are integrated into the wearable sensor platform 10 by the manufacturer and may not be intended to be removed by the end user. The wearable sensor platform 10 may be waterproof or water sealed.

The band or strap 12 may be one piece or modular. The band 12 may be made of a fabric. For example, a wide range of twistable and expandable elastic mesh/textiles are contemplated. The band 12 may also be configured as a multi-band or in modular links. The band 12 may include a latch or a clasp mechanism to retain the watch in place in certain implementations. In certain embodiments, the band 12 will contain wiring (not shown) connecting, among other things, the base module 18 and sensor module 16. Wireless communication, alone or in combination with wiring, between base module 19 and sensor module 16 is also contemplated.

The sensor module 16 may be removably attached on the band 12, such that the sensor module 16 is located at the bottom of the wearable sensor platform 10 or, said another way, on the opposite end of the base module 18. Positioning the sensor module 16 in such a way to place it in at least partial contact with the skin on the underside of the user's wrist to allow the sensor units 28 to sense physiological data from the user. The contacting surface(s) of the sensor units 28 may be positioned above, at or below, or some combination such positioning, the surface of the sensor module 16.

The base module 18 attaches to the band 12 such that the base module 18 is positioned at top of the wearable sensor platform 10. Positioning the base module 18 in such a way to place it in at least partial contact with the top side of the wrist.

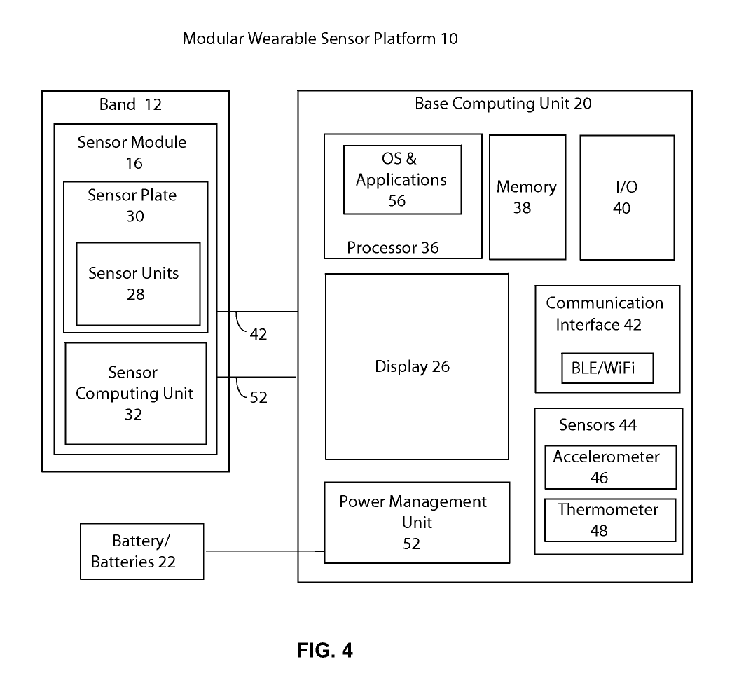

The base module 18 may include a base computing unit 20 and a display 26 on which a graphical user interface (GUI) may be provided. The base module 18 performs functions including, for example, displaying time, performing calculations and/or displaying data, including sensor data collected from the sensor module 16. In addition to communication with the sensor module 16, the base module 18 may wirelessly communicate with other sensor module(s) (not shown) worn on different body parts of the user to form a body area network, or with other wirelessly accessible devices (not shown), like a smartphone, tablet, display or other computing device. As will be discussed more fully with respect to FIG. 4, the base computing unit 20 may include a processor 36, memory 38, input/output 40, a communication interface 42, a battery 22 and a set of sensors 44, such as an accelerometer/gyroscope 46 and thermometer 48.

The sensor module 16 collects data (e.g., physiological, activity data, sleep statistics and/or other data), from a user and is in communication with the base module 18. The sensor module 16 includes sensor units 28 housed in a sensor plate 30. For certain implementations, because a portable device, such as a wristwatch, has a very small volume and limited battery power, sensor units 28 of the type disclosed may be particularly suited for implementation of a sensor measurement in a wristwatch. In some embodiments, the sensor module 16 is adjustably attached to the band 12 such that the base module 18 is not fixedly positioned, but can be configured differently depending on the physiological make-up of the wrist.

The sensor units 28 may include an optical sensor array, a thermometer, a galvanic skin response (GSR) sensor array, a bioimpedance (BioZ) sensor array, an electrocardiogram (ECG) sensor, or any combination thereof. The sensors units 28 may take information about the outside world and supply it to the wearable modular sensor platform 10. The sensors 28 can also function with other components to provide user or environmental input and feedback to a user. For example, a MEMS accelerometer may be used to measure information such as position, motion, tilt, shock, and vibration for use by processor 36. Other sensor(s) may also be employed. The sensor module 16 may also include a sensor computing unit 32. The sensor units 28 may also include biological sensors (e.g., pulse, pulse oximetry, body temperature, blood pressure, body fat, etc.), proximity detector for detecting the proximity of objects, and environmental sensors (e.g., temperature, humidity, ambient light, pressure, altitude, compass, etc.).

In other embodiments, the clasp 34 also provides an ECG electrode. One or more sensor units 28 and the ECG electrode on the clasp 34 can form a complete ECG signal circuit when the clasp 34 is touched. The sensor computing unit 32 may analyze data, perform operations (e.g., calculations) on the data, communicate data and, in some embodiments, may store the data collected by the sensor units 28. In some embodiments, the sensor computing unit 32 receives (for example, data indicative of an ECG signal) from one or more of the sensors of the sensor units 28, and processes the received data to form a predefined representation of a signal (for example, an ECG signal).

The sensor computing unit 32 can also be configured to communicate the data and/or a processed form of the received data to one or more predefined recipients, for example, the base computing unit 20, for further processing, display, communication, and the like. For example, in certain implementations the base computing unit 20 and/or sensor computing unit determine whether data is reliable and determine an indication of confidence in the data to the user.

Because the sensor computing unit 32 may be integrated into the sensor plate 30, it is shown by dashed lines in FIG. 1. In other embodiments, the sensor computing unit 32 may be omitted or located elsewhere on the wearable sensor platform 10 or remotely from the wearable sensor platform 10. In an embodiment where the sensor computing unit 32 may be omitted, the base computing unit 20 may perform functions that would otherwise be performed by the sensor computing unit 32. Through the combination of the sensor module 16 and base module 18, data may be collected, transmitted, stored, analyzed, transmitted and presented to a user.

The wearable sensor platform 10 depicted in FIG. 1 is analogous to the wearable sensor platform 10 depicted in FIGS. 2 and 3. Thus, the wearable sensor platform 10 includes a band 12, a battery 22, a clasp 34, a base module 18 including a display/GUI 26, a base computing unit 20, and a sensor module 16 including sensor units 28, a sensor plate 30, and an optional sensor computing unit 32. However, as can be seen in FIG. 3, the locations of certain modules have been altered. For example, the clasp 34 is closer in FIG. 3 to the display/GUI 26 than clasp 34 is in FIG. 1. Similarly, in FIG. 3, the battery 22 is housed with the base module 18. In the embodiment shown in FIG. 1, the battery 22 is housed on the band 12, opposite to the display 26. However, it should be understood that, in some embodiments, the battery 22 charges the base module 18 and optionally an internal battery (not shown) of the base module 18. In this way, the wearable sensor platform 10 may be worn continuously. Thus, in various embodiments, the locations and/or functions of the modules and other components may be changed.

FIG. 3 is a diagram illustrating one embodiment of a modular wearable sensor platform 10 and components comprising the base module 18. The wearable sensor platform 10 is analogous to the wearable sensor platform 10 in FIGS. 1 and 2 and thus includes analogous components having similar reference labels. In this embodiment, the wearable sensor platform 10 may include a band 12, and a sensor module 16 attached to band 12. The removable sensor module 16 may further include a sensor plate 30 attached to the band 12, and sensor units 28 attached to the sensor plate 30. The sensor module 16 may also include a sensor computing unit 32.

The wearable sensor platform 10 includes a base computing unit 20 in FIG. 3 analogous to the base computing unit 20 and one or more batteries 22 in FIG. 3. For example, permanent and/or removable batteries 22 that are analogous to the battery 22 in FIGS. 1 and 2 may be provided. In one embodiment, the base computing unit 20 may communicate with or control the sensor computing unit 32 through a communication interface 42. In one embodiment, the communication interface 42 may comprise a serial interface. The base computing unit 20 may include a processor 36, a memory 38, input/output (I/O) 40, a display 26, a communication interface 42, sensors 44, and a power management unit 52.

The processor 36, the memory 38, the I/O 40, the communication interface 42 and the sensors 44 may be coupled together via a system bus (not shown). The processor 36 may include a single processor having one or more cores, or multiple processors having one or more cores. The processor 36 may be configured with the I/O 40 to accept, receive, transduce and process verbal audio frequency command, given by the user. For example, an audio codec may be used. The processor 36 may execute instructions of an operating system (OS) and various applications 56. The processor 36 may control on command interactions among device components and communications over an I/O interface. Examples of the OS 56 may include, but not limited to, Linux Android™, and Android Wear.

The memory 38 may comprise one or more memories comprising different memory types, including RAM (e.g., DRAM and SRAM) ROM, cache, virtual memory microdrive, hard disks, microSD cards, and flash memory, for example. The I/O 40 may comprise a collection of components that input information and output information. Example components comprising the I/O 40 having the ability to accept inputted, outputted or other processed data include a microphone, messaging, camera and speaker. I/O 40 may also include an audio chip (not shown), a display controller (not shown), and a touchscreen controller (not shown).

The communication interface 42 may include components for supporting one-way or two-way wireless communications and may include a wireless network interface controller (or similar component) for wireless communication over a network in some implementations, a wired interface in other implementations, or multiple interfaces. In one embodiment, the communication interface 42 is for primarily receiving data remotely, including streaming data, which is displayed and updated on the display 26. However, in an alternative embodiment, besides transmitting data, the communication interface 42 could also support voice transmission. In an exemplary embodiment, the communication interface 42 supports low and intermediate power radio frequency (RF) communications. In certain implementations, example types of wireless communication may include Bluetooth Low Energy (BLE), WLAN (wireless local area network), WiMAX, passive radio-frequency identification (RFID), network adapters and modems. However, in another embodiment, example types of wireless communication may include a WAN (Wide Area Network) interface, Wi-Fi, WPAN, multi-hop networks, or a cellular network such as 3G, 4G, 5G or LTE (Long Term Evolution). Other wireless options may include ultra-wide band (UWB) and infrared, for example. The communication interface 42 may also include other types of communications devices (not shown) besides wireless, such as serial communications via contacts and/or USB communications. For example, a micro USB-type USB, flash drive, or other wired connection may be used with the communication interface 42.

In one embodiment, the display 26 may be integrated with the base computing unit 20; while in another embodiment, the display 26 may be external from the base computing unit 20. Display 26 may be flat or curved, e.g., curved to the approximate curvature of the body part on which the wearable sensor module platform 10 is located (e.g., a wrist, an ankle, a head, etc.).

Display 26 may be a touch screen or gesture controlled. The display 26 may be an OLED (Organic Light Emitting Diode) display, TFT LCD (Thin-Film-Transistor Liquid Crystal Display), or other appropriate display technology. The display 26 may be active-matrix. An example display 26 may be an AMOLED display or SLCD. The display may be 3D or flexible. The sensors 44 may include any type of microelectromechanical systems (MEMs) sensor. Such sensors may include an accelerometer/gyroscope 46 and a thermometer 48, for instance.

The power management unit 52 may be coupled to the power source 22 and may comprise a microcontroller that communicates and/or controls power functions of at least the base computing unit 20. Power management unit 52 communicates with the processor 36 and coordinates power management. In some embodiments, the power management unit 52 determines if a power level falls below a certain threshold level. In other embodiments, the power management unit 52 determines if an amount of time has elapsed for secondary charging.

The power source 22 may be a permanent or removable battery, fuel cell or photo voltage cell, etc. The battery 22 may be disposable. In one embodiment, the power source 22 may comprise a rechargeable, lithium ion battery or the like may be used, for example. The power management unit 52 may include a voltage controller and a charging controller for recharging the battery 22. In some implementations, one or more solar cells may be used as a power source 22. The power source 22 may also be powered or charged by AC/DC power supply. The power source 22 may charge by non-contact or contact charging. In one embodiment, the power management unit 52 may also communicate and/or control the supply of battery power to the removable sensor module 16 via power interface 52. In some embodiments, the battery 22 is embedded in the base computing unit 20. In other embodiments, the battery 22 is external to the base computing unit 20.

Other wearable device configurations may also be used. For example, the wearable sensor module platform can be implemented as a leg or arm band, a chest band, a wristwatch, an article of clothing worn by the user such as a snug fitting shirt, or any other physical device or collection of devices worn by the user that is sufficient to ensure that the sensor units 28 are in contact with approximate positions on the user's skin at a particular measurement site to obtain accurate and reliable data.

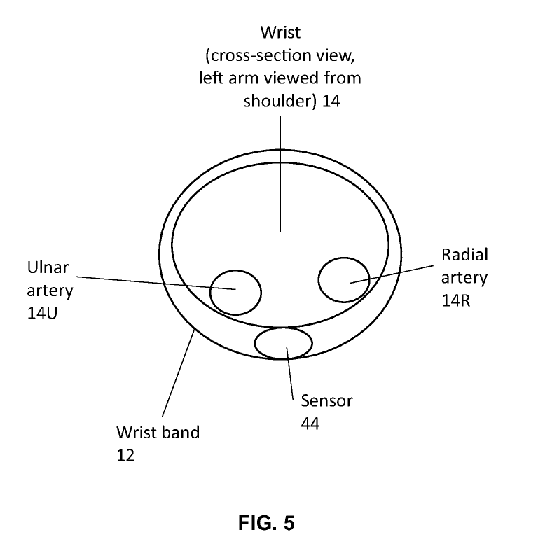

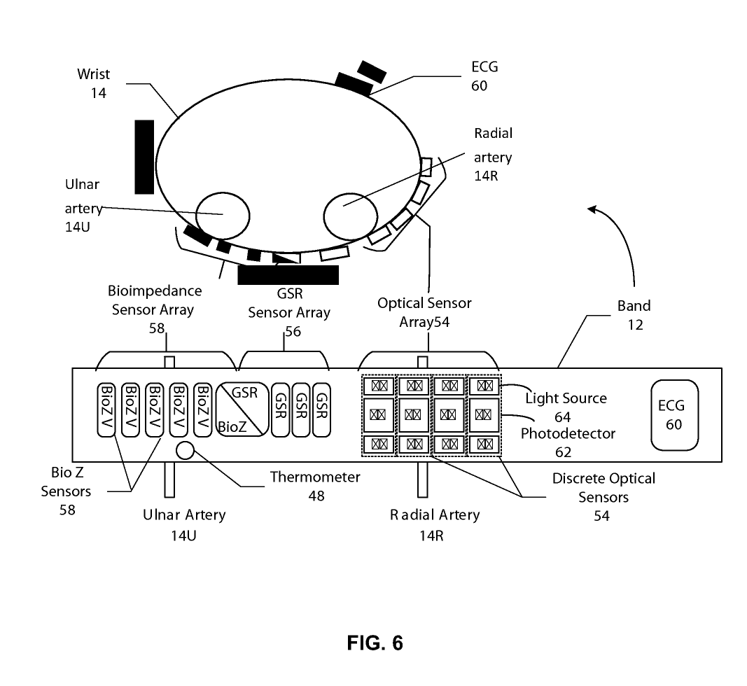

FIG. 5 is a diagram of a cross section of a wrist 14. More specifically, by way of example, FIG. 6 is a diagram illustrating an implementation of a wearable sensor module 10. The top portion of FIG. 6 illustrates the wearable sensor module 10 wrapped around a cross-section of a user's wrist 14, while the bottom portion of FIG. 6 shows the band 12 in an flattened position.

According to this embodiment, the wearable sensor module 10 includes at least an optical sensor array 54, and may also include optional sensors, such as a galvanic skin response (GSR) sensor array 56, a bioimpedance (BioZ) sensor array 58, and an electrocardiogram (ECG) sensor 60, or any combination of which may comprise a sensor array.

According to another embodiment, the sensor units 28 configured as a sensor array(s) comprising an array of discrete sensors that are arranged or laid out on the band 12, such that when the band 12 is worn on a body part, each sensor array may straddle or otherwise address a particular blood vessel (i.e., a vein, artery, or capillary), or an area with higher electrical response irrespective of the blood vessel.

More particularly, as can be seen in FIGS. 5 and 6, the sensor array may be laid out substantially perpendicular to a longitudinal axis of the blood vessel (e.g., radial artery 14R and/or ulnar artery 14U) and overlaps a width of the blood vessel to obtain an optimum signal. In one embodiment, the band 12 may be worn so that the sensor units 28 comprising the sensor array(s) contact the user's skin, but not so tightly that the band 12 is prevented from any movement over the body part, such as the user's wrist 14, or creates discomfort for the user at sensor contact points.

In another embodiment, the sensor units 28 may comprise an optical sensor array 54 that may comprise a photoplethysmograph (PPG) sensor array that may measures relative blood flow, pulse and/or blood oxygen level. In this embodiment, the optical sensor array 54 may be arranged on sensor module 16 so that the optical sensor array 54 is positioned in sufficient proximity to an artery, such as the radial or ulnar artery, to take adequate measurements with sufficient accuracy and reliability.

Further details of the optical sensor array 54 will now be discussed. In general, configuration and layout of each of the discrete optical sensors 54 may vary greatly depending on use cases. In one embodiment, the optical sensor array 54 may include an array of discrete optical sensors 54, where each discrete optical sensor 54 is a combination of at least one photodetector 62 and at least two matching light sources 64 located adjacent to the photodetector 62. In one embodiment, each of the discrete optical sensors 54 may be separated from its neighbor on the band 12 by a predetermined distance of approximately 0.5 to 2 mm.

In one embodiment, the light sources 64 may each comprise a light emitting diode (LED), where LEDs in each of the discrete optical sensors emit light of a different wavelength. Example light colors emitted by the LEDs may include green, red, near infrared, and infrared wavelengths. Each of the photodetectors 62 convert received light energy into an electrical signal. In one embodiment, the signals may comprise reflective photoplethysmograph signals. In another embodiment, the signals may comprise transmittance photoplethysmograph signals. In one embodiment, the photodetectors 62 may comprise phototransistors. In alternative embodiment, the photodetectors 62 may comprise charge-coupled devices (CCD).

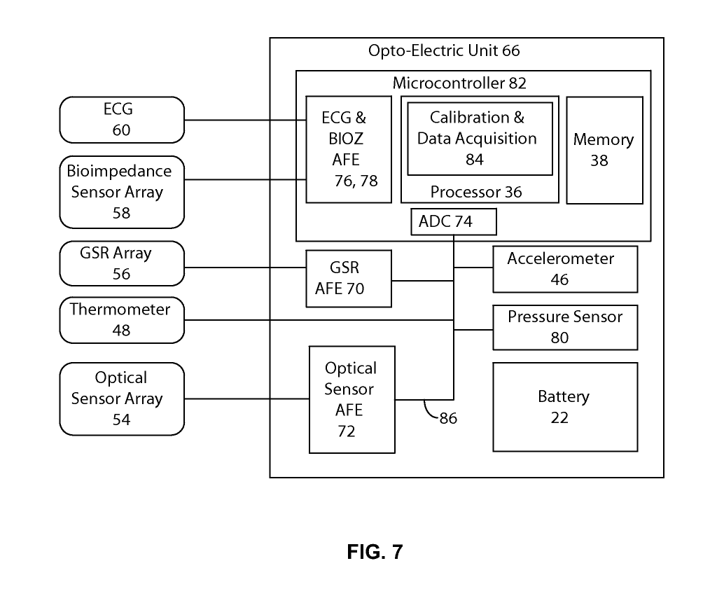

FIG. 7 is a block diagram illustrating another configuration for components of wearable sensor module in a further implementation. In this implementation, the ECG 60, the bioimpedance sensor array 58, the GSR array 56, the thermometer 48, and the optical sensor array 54 may be coupled to an optical-electric unit 66 that controls and receives data from the sensors on the band 12. In another implementation, the optical-electric unit 66 may be part of the band 12. In an alternative implementation, the optical-electric unit 66 may be separate from the band 12.

The optical-electric unit 66 may comprise an ECG and bioimpedance (BIOZ) analog front end (AFE) 76, 78, a GSR AFE 70, an optical sensor AFE 72, a processor 36, an analog-to-digital converter (ADC) 74, a memory 38, an accelerometer 46, a pressure sensor 80 and a power source 22.

As used herein, an AFE 68 may comprise an analog signal conditioning circuitry interface between corresponding sensors and the ADC 74 or the processor 36. The ECG and BIOZ AFE 76, 78 exchange signals with the ECG 60 and the bioimpedance sensor array 58. The GSR AFE 70 may exchange signals with the GSR array 56 and the optical sensor AFE 72 may exchange signals with the optical sensor array 54. In one embodiment, the GSR AFE 70, the optical sensor AFE 72, the accelerometer 46, and the pressure sensor 80 may be coupled to the ADC 74 via bus 86. The ADC 74 may convert a physical quantity, such as voltage, to a digital number representing amplitude.

In one embodiment, the ECG and BIOZ AFE 76, 78, memory 38, the processor 36 and the ADC 74 may comprise components of a microcontroller 82. In one embodiment, the GSR AFE 70 and the optical sensor AFE 72 may also be part of the microcontroller 82. The processor 36 in one embodiment may comprise a reduced instruction set computer (RISC), such as a Cortex 32-bit RISC ARM processor core by ARM Holdings, for example.

According to an exemplary embodiment, the processor 36 may execute a calibration and data acquisition component 84 that may perform sensor calibration and data acquisition functions. In one embodiment, the sensor calibration function may comprise a process for self-aligning one more sensor arrays to a blood vessel. In one embodiment, the sensor calibration may be performed at startup, prior to receiving data from the sensors, or at periodic intervals during operation.

In another embodiment, the sensor units 28 may also comprise a galvanic skin response (GSR) sensor array 56, which may comprise four or more GSR sensors that may measure electrical conductance of the skin that varies with moisture level. Conventionally, two GSR sensors are necessary to measure resistance along the skin surface. According to one aspect of this embodiment, the GSR sensor array 56 is shown including four GSR sensors, where any two of the four may be selected for use. In one embodiment, the GSR sensors 56 may be spaced on the band 2 to 5 mm apart.

In another embodiment, the sensor units 28 may also comprise bioimpedance (BioZ) sensor array 58, which may comprise four or more BioZ sensors 58 that measure bioelectrical impedance or opposition to a flow of electric current through the tissue. Conventionally, only two sets of electrodes are needed to measure bioimpedance, one set for the “I” current and the other set for the “V” voltage. However, according to an exemplary embodiment, a bioimpedance sensor array 58 may be provided that includes at least four to six bioimpedance sensors 58, where any four of electrodes may be selected for “I” current pair and the “V” voltage pair. The selection could be made using a multiplexor. In the embodiment shown, the bioimpedance sensor array 58 is shown straddling an artery, such as the Radial or Ulnar artery. In one embodiment, the BioZ sensors 58 may be spaced on the band 5 to 13 mm apart. In one embodiment, one or more electrodes comprising the BioZ sensors 58 may be multiplexed with one or more of the GSR sensors 56.

In yet another embodiment, the band 12 may include one or more electrocardiogram (ECG) sensors 60 that measure electrical activity of the user's heart over a period of time. In addition, the band 12 may also comprise a thermometer 48 for measuring temperature or a temperature gradient.

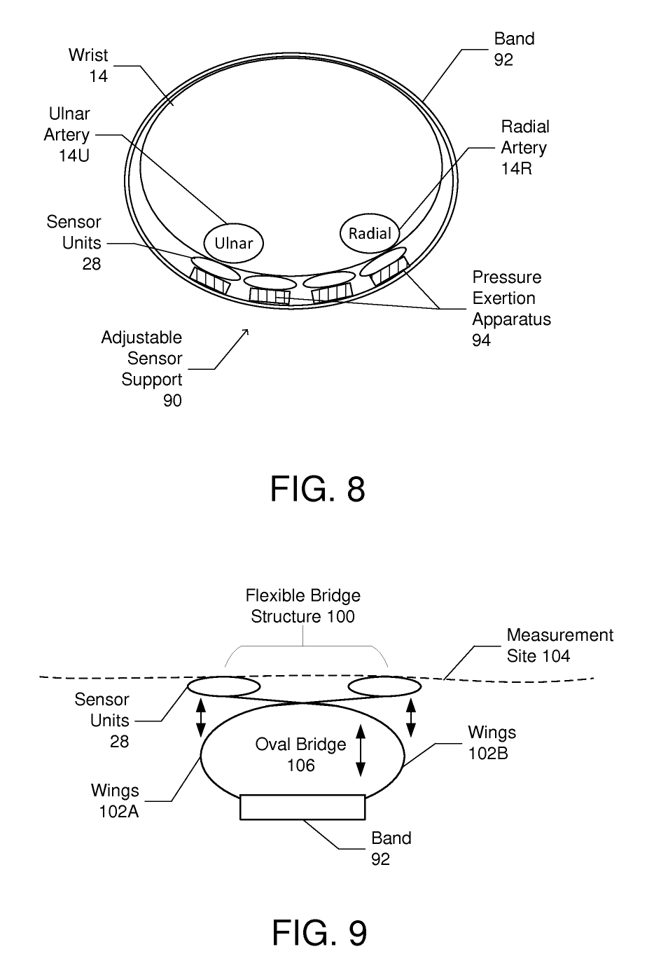

FIG. 8 is a diagram of a cross section of a wrist 14 and an embodiment of an adjustable sensor support structure 90 according to a further aspect of the exemplary embodiment. In one embodiment, the adjustable sensor support structure 90 may include one or more sensor arrays having a plurality of sensor units 28 arranged on a band 92 such that the sensor array straddles or otherwise addresses a blood vessel when worn on a measurement site of a user, as described above.

According to the exemplary embodiments, the adjustable sensor support structure 90 further includes a pressure exertion apparatus 94 attached between the sensor units 28 and the band 92 that exerts outward pressure on the sensor units 28 towards the measurement site causing the sensor units 28 to maintain contact with skin of the user independent of motion activity of the band 12, thereby improving contact quality.

According to exemplary embodiments, adjustability of the pressure exertion apparatus 94 may be provided through the use of multiple embodiments, including at least one of; a flexible bridge structure, a flexible foam structure, and a sensor trampoline structure.

In the first embodiment, the flexible bridge structure may have various allowed degrees of freedom allowing, for example, folding and multiple flex points. This embodiment includes folding material into 3D bridge structures that compress and flex in two dimensions. The second embodiment may include a flexible foam structure/material formed in a desired topology with or without springs that aid in supporting and adjusting the sensor units 28. In the third embodiment, the pressure exertion apparatus 94 may comprise a sensor trampoline structure. In each embodiment, the pressure exertion apparatus 94 may comprise various support structures and/or materials designed such that the sensor units 28 are allowed to be pushed closer to or farther away from the measurement site.

When the band 92 with the pressure exertion apparatus 94 is worn about a measurement site of a user, such as the wrist 14, varying topology of the wrist 14 may cause force(s) to simultaneously be exerted upon the pressure exertion apparatus 94 due to compliance of the band 92 to the varying topology of the wrist 14. Therefore, the pressure exertion apparatus 94 compensates for varying topology of the measurement site by adjusting the sensor array and/or the individual sensor units 28 in the measurement plane normal direction. In one embodiment, individual sensor units 28 are independently adjustable from one another.

In one embodiment the measurement site may be largely planar in a spatially constrained local area, but may have varying topology across a larger spatial area. In accordance with one embodiment, the sensor units 28 corresponding to different measurement site areas may be configured with individual pressure exertion apparatuses such that the sensor units 28 in the individual measurement site areas are mechanically decoupled from one another and independently adjustable in z-height.

Although the exemplary embodiment is described as a sensor array attached to the pressure exertion apparatus 94, in another embodiment, the pressure exertion apparatus 94 may be attached to a single sensor unit 28, or to several sensor units 28 not laid out in an array.

FIG. 9 is a diagram is a diagram of a cross section of the band 92 and an embodiment where the pressure exertion apparatus 90 comprises a flexible bridge structure 100. In one embodiment, the flexible bridge structure 100 comprises two bendable wings, a first wing 102A and a second wing 102B (collectively referred to as wings 102). One end of the first wing 102A is attached to one side of the band 92 and one end of the second wing 102B is attached to an opposite side of the band 92. Open ends of each of the wings 102 are folded back on one another to form an oval bridge 106, where the open ends of both wings 102 are free hanging over the oval bridge 106. At least one sensor unit 28 (e.g., an electrode) may be attached to the open ends of at least one of the wings 102, although preferably a sensor unit 28 is attached to both wings as shown. Such a flexible bridge structure 100 may form a substantially convex oval bridge 106 with the sensor units 28 being placed on the convex side of the oval bridge 106 facing the measurement site 104.

According to this embodiment, the flexible bridge structure 100 is configured to enable multi-dimensional flexing. When an amount of force above a threshold amount is exerted upon the wings 102 on the convex side of the oval bridge 106, the flexible bridge structure 100 may respond by compressing and flexing wider in order to accommodate the exerted force.

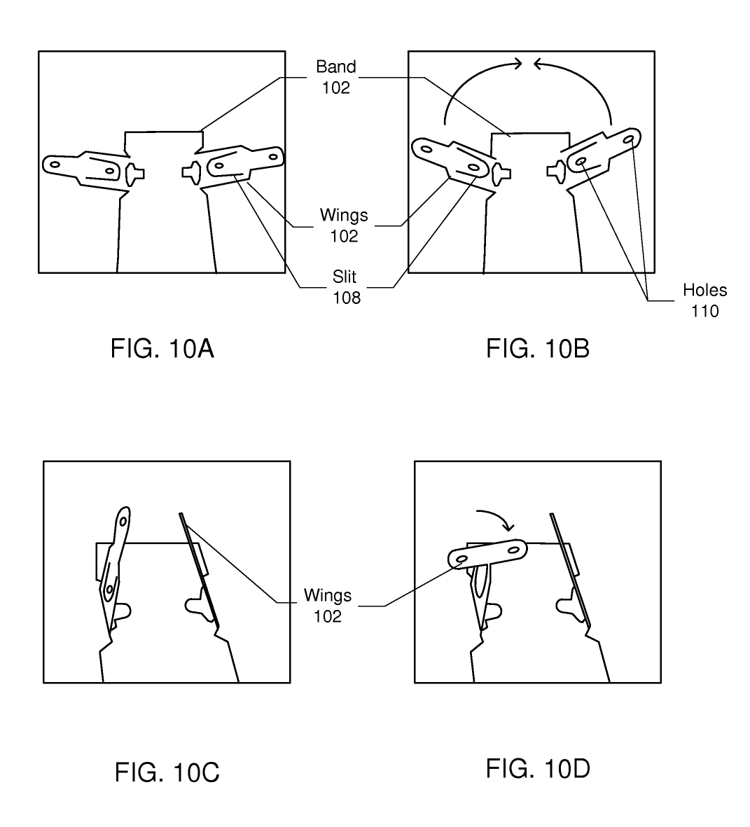

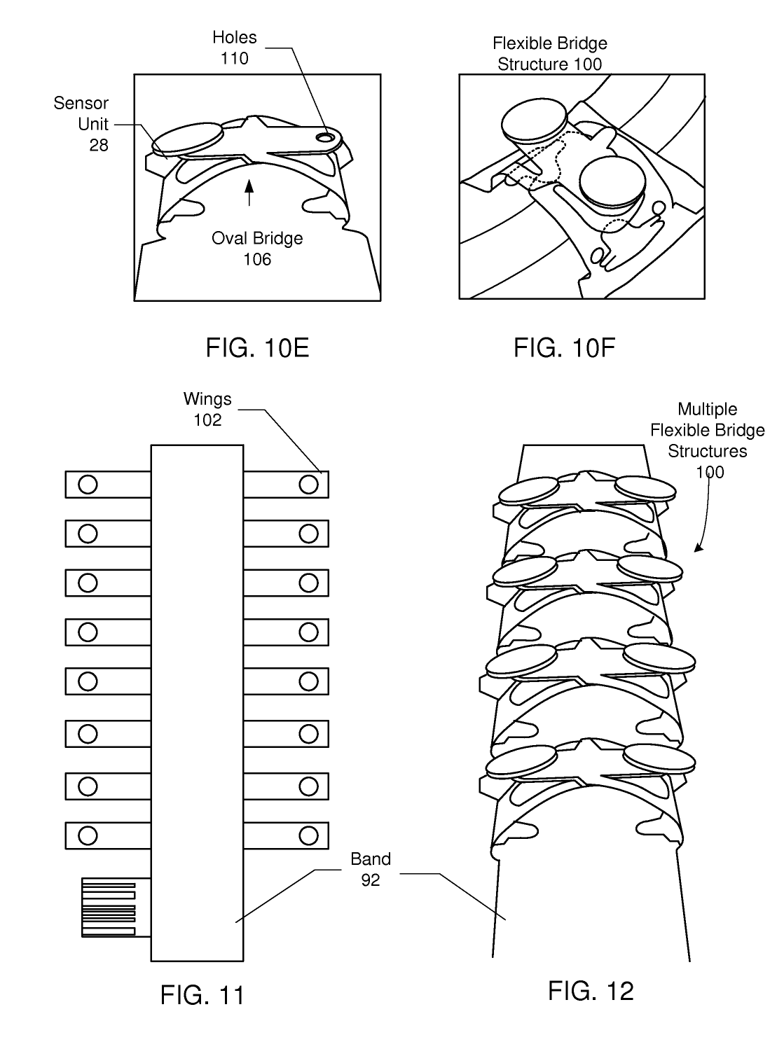

FIGS. 10A through 10F illustrate an exemplary process for assembling the flexible bridge structure 100. FIG. 10A shows the process may include at least one of printing and/or etching circuitry onto a plastic film that may form at least part of the band 92, and laser cutting the plastic film to form a shape of two-stage foldable wings 102. The wings 102 are also formed with a slit 108 that is used to form an opening in the wings 102 and holes 110 for affixing the sensor units 28. In the embodiment shown, the slit 108 may be cut in the shape of a “U”, although other shapes are also possible.

FIGS. 10B and 10C show a first folding stage where the wings 102 are folded upwards away from the band 92 such that the wings stand approximately perpendicular to the band 92.

FIG. 10D shows a seconds folding stage where the open end of the wings 102 are folded downward towards the band 92, causing the open end of the wings and material formed by the slit 108 to lie approximately parallel to the band 92.

FIG. 10E shows that the open ends of each of the wings 102 are inserted into the “U” shaped slit 108 in the opposite wing so that the wings 102 align on top of each other, e.g. via the respective holes 110, thereby forming the oval bridge 106 that bends and flexes under pressure. The wings may be held or fastened together with some a type of fastener, e.g., glue, staples and the like.

The sensor units 28 are then affixed to the oval bridge 106, e.g., through the holes 110 in the wings 102, forming a seesaw-like structure that sits atop the oval bridge 106, as shown in FIG. 10F. In one embodiment, the sensor units 28 may be printed on, be part of, or otherwise attached to the wings 102. In one embodiment, the sensor units 28 themselves may form a fasting mechanism that holds the wings 102 together.

The completed flexible bridge structure 100 is multidimensional in that the wings 102 may move up and down independently (2D flex), while the oval bridge 106 compresses and flexes as well (2D flex). Several methods may be utilized in order to optimize the functionality of the bridge formation including, but not limited to, adjustment of bridge length, adjustment of material properties (i.e. thickness, elastic modulus, etc.), and addition of support material(s) to encapsulate the bridge (e.g., thermoplastic polyurethanes, closed foam material, etc.).

According to a further embodiment, the adjustable sensor support structure may comprise multiple flexible bridge structures 100.

FIGS. 11 and 12 are diagrams illustrating one embodiment of multiple flexible bridge structures 100 connected edge-to-edge on the band 92 in a serial configuration. FIG. 11 shows the wings 102 and the band 92 in a flat position after printing, etching and cutting. And FIG. 12 shows the multiple flexible bridge structures 100 after the wings 102 have been bent into the oval bridges 106 and the sensor units 28 affixed to each of the oval bridges 106. In one embodiment, the flexible bridge structures 100 may cover only a portion of the band 92, while in another embodiment, the flexible bridge structures 100 may cover substantially all of the band 92 and cover most, if not all, of the wrist.

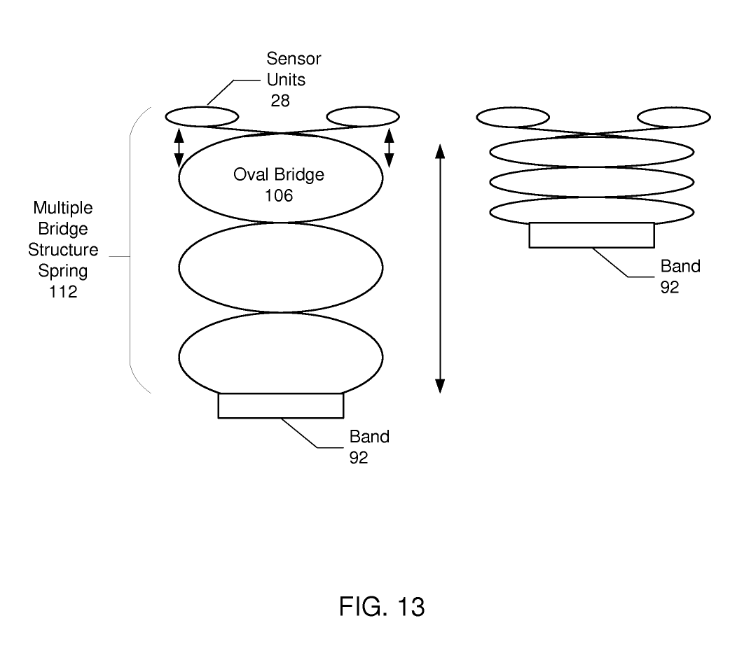

FIG. 13 is a diagram illustrating yet another embodiment of the multiple flexible bridge structures 100 in which the flexible bridge structures are layered on top of each other to form a multiple bridge structure spring 112. In the example shown, three oval bridges 106 are formed with both the wings and sensor units 28 on top of the multiple bridge structure spring 112. The multiple bridge structure spring 112 adjust height and compresses, causing the sensor units 28 to push against the skin and reduce motion artifacts.

According to the exemplary embodiments, each of the flexible bridge structures 100 and 112 may flex independently from one another, and each of the sensor units 28 thereon may also flex independently. The flexible bridge structures 102 naturally take the shape with least stress on the structure, thus inherently the flexible bridge structures 102 are in an “expanded” position. Force due to compliance with the wrist shape compresses the flexible bridge structures 102, and thus creates a reactive force pressing outward toward the measurement site. Variations in the shape of the limb relative to the band 92 (upon movements of the limb) is nivellated by the flexible bridge structures 100, which respond to shape changes. Thus, the flexible bridge structures 100 take care of the relative displacement of the limb to the band 92 by adjusting bridge height.

As described above, another embodiment the pressure exertion apparatus is a flexible foam structure. In one embodiment, the flexible foam structure may comprise foam islands, while in another embodiment the flexible foam structure may comprise flexible cavity structures.

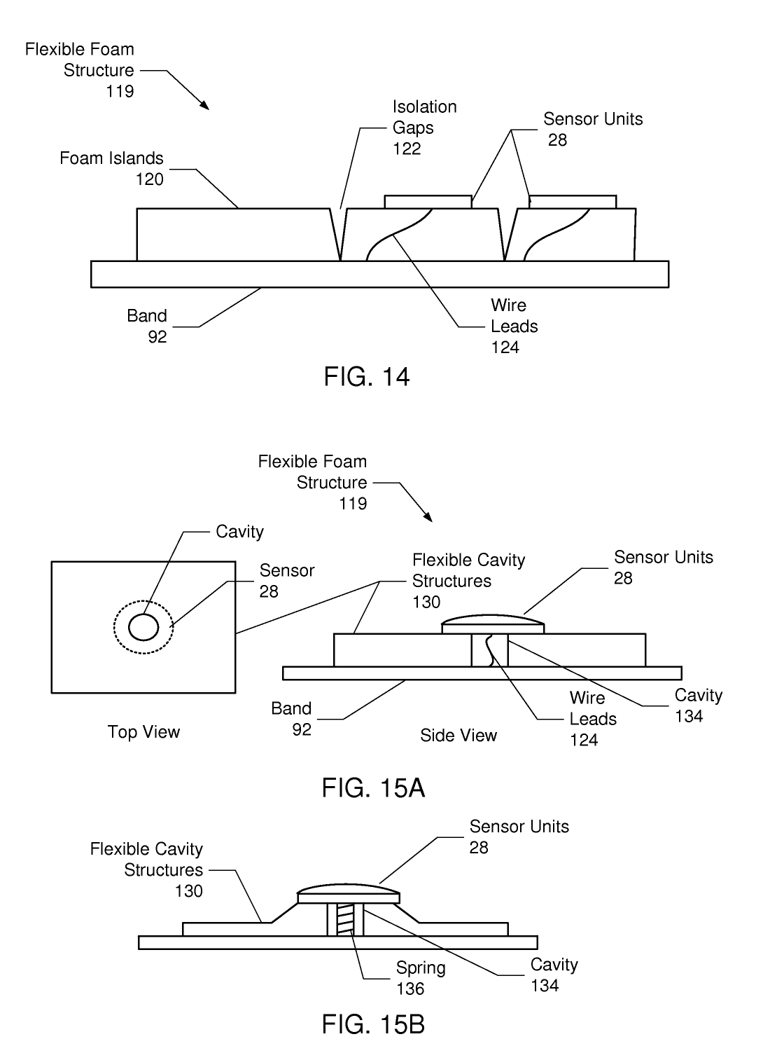

FIG. 14 is a diagram is a diagram of a cross section of the band 92 showing the embodiment where the flexible foam structure 119 comprises foam islands 120. The flexible foam structures 119 may comprise a plurality of foam islands 120 that are mounted upon a band 92, where at least a portion of each of the foam islands 120 support at least one sensor unit 28. According to one embodiment, isolation gaps 120 are formed between at least portions of the foam islands 120 that are created from a shape of foam islands 120 to allow expansion of the foam islands 120 upon application of force to the sensor units 28. The band 92 may comprise a flexible printed circuit board (PCB) and wire leads 124 may be inserted through the foam island structures 120 connecting the sensor units 28 to the band 92. In one embodiment, the foam islands 120 may be constructed from, for example, a foam and/or a thermoplastic elastomer, such as thermoplastic polyurethane.

FIGS. 15A and 15B are diagrams of a cross section of the band 92 showing the embodiment where the flexible foam structure 119 comprises flexible cavity structures 132. The flexible cavity structures 132 may comprise elastomer or foam that are formed in a desired topology on the band 92, where at least a portion of flexible cavity structures 132 support at least one sensor unit 28. According to one embodiment, internal cavities 134 are in the formed in the flexible cavity structures 130 that enable expansion volume upon compression of the flexible cavity structures 130 upon application of force to the sensor units 28. Wire leads 124 may be placed in the internal cavities 134 connecting the sensor units 28 to the band 92.

FIG. 15B is a diagram illustrating a further embodiment of the flexible cavity structures 130. In this embodiment, the flexible cavity structures 130 may be formed . . . and the cavity 134 thereof may contain a spring 136 that is configured to provide further elastic support to the flexible cavity structures 130 and/or to transmit an electrical signal between the sensor units 28 and the band 92.

Also shown in FIG. 15B, in both the foam island 120 and flexible cavity structures 130 embodiments and other related structural configurations of flexible foam structure, the elasticity of the flexible foam structure 119 may be adjusted by tuning the ratio of expansion volume to the contact area.

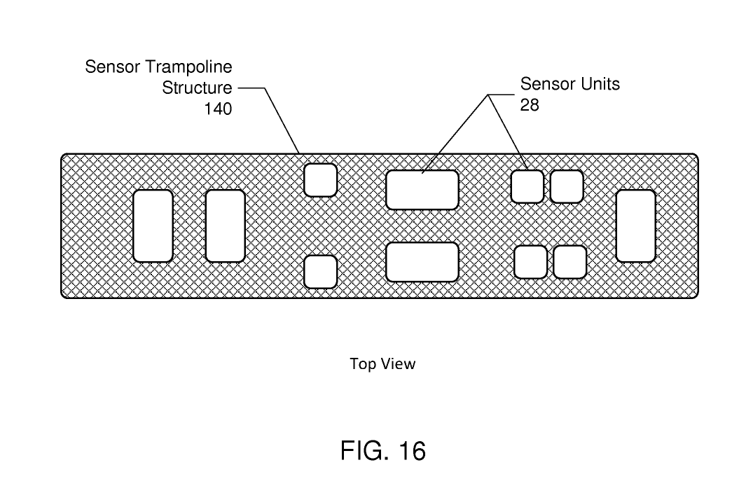

FIG. 16 is a diagram illustrating an embodiment where the pressure exertion apparatus 94 of the adjustable sensor support structure 90 comprises a sensor trampoline structure 140. According to an embodiment of the present invention, the sensor trampoline structure 140 may comprise a multi-dimensional spring-like mesh that supports multiple sensor units 28. The sensor trampoline structure 140 may be substantially constructed from wire and exhibit spring tension in multiple dimensions to provide elastic support to the sensor units 28. In one embodiment, the sensor trampoline structure 140 allows the sensor units 28 to move independently in z-height, while twisting from side-to-side, if necessary, upon application of force to the sensor units 28. The sensor trampoline structure 140 may be configured to and/or to transmit an electrical signal to/from the sensor units 28 to the base 92. In one embodiment, the sensor trampoline structure 140 may be attached to the base 92 or used in place of the base 92.

Additionally, in all the adjustable sensor support structure embodiments, the band 92 may contain additional circuitry and/or functional electrical components including but not limited to an application specific integrated circuit, a field programmable grid array, a battery, a microcontroller, or others.

The embodiments above described mainly passive mechanisms of adjustability of the sensors in height and planarity where materials comprising the adjustable sensor support structure may be chosen on the basis of compressibility, the thermal expansion coefficients and to optimize the forces exerted on the sensor units 28 as a response to a physical change like the presence of a shape of body part.

However, according to a further embodiment, the adjustable sensor support structure may be provided with an active adjustment mechanism. In one embodiment, responsive to the sensor units 28 detecting a physiologic signal from the body (ECG, PPG, bioimpedance, galvanic skin response and alike), the quality of this signal can be received by the active adjustment mechanism and used to actively optimize sensor unit contact with the body. As an example, the active adjustment mechanisms may be used to push the sensor units 28 toward the body to improve skin contact based on the signal quality. The resulting feedback loop may optimize the topology of the sensor units 28 in height and planarity, and reduce the motion artifacts provided the speed of the adjustment is aligned with the necessary speed in adjustments of the topology. In one embodiment the active adjustment mechanisms may include solenoids, mechanically driving mini-servo motors or hydraulically.

This active intelligent method to adjust the sensor units 28 will also yield valuable information on the dynamic nature of skin and sensor contact in active use. This information can be used in the artifact reduction during data processing of the obtained sensor signals in addition to information from accelerometers, gyroscopes or GPS.

In a further embodiment, the active adjustment mechanism may cause optimal sensor topology settings for a particular user/body part to be saved, wherein the optimal sensor topology settings may be used to identify the wearer of the adjustable sensor support structure, as body contours are similar to fingerprints.

Other kinds of devices can be used to provide interaction with a user as well. For example, the adjustable sensor support structure may provide to the user any form of sensory feedback (e.g., visual feedback, auditory feedback, or tactile feedback); and the adjustable sensor support structure may receive input from the user in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other. Various cloud-based platforms and/or other database platforms may be employed in certain implementations of the modular sensor platform 10 to, for example, receive and send data to the modular sensor platform 10. One such implementation is architecture for multi-modal interactions (not shown). Such architecture can be employed as a layer of artificial intelligence between wearable devices, like modular sensor platform 10, and the larger cloud of other devices, websites, online services, and apps. Such an architecture also may serve to translate (for example by monitoring and comparing) data from the modular sensor platform 10 with archived data, which may be then be used to alert, for example, the user or healthcare professional about changes in condition. This architecture further may facilitate interaction between the modular sensor platform 10 and other information, such as social media, sports, music, movies, email, text messages, hospitals, prescriptions to name a few.

A method and system for providing an adjustable sensor support structure has been disclosed. The present invention has been described in accordance with the embodiments shown, and there could be variations to the embodiments, and any variations would be within the spirit and scope of the present invention. Accordingly, many modifications may be made by one of ordinary skill in the art without departing from the spirit and scope of the appended claims.

We claim:

1. An adjustable sensor support structure, comprising:
a sensor array comprising a plurality of sensor units arranged on a band such that the sensor array straddles or otherwise addresses a blood vessel when worn on a measurement site of a user; and
a pressure exertion apparatus attached between the sensor units and the band that exerts outward pressure on the sensor units towards the measurement site causing the sensor units to maintain contact with skin of the user independent of motion activity of the band, thereby improving contact quality,
wherein the pressure exertion apparatus comprises at least one of:
a flexible bridge structure,
a flexible foam structure, and
a sensor trampoline structure.

2. The adjustable sensor support structure of claim 1, wherein the sensor units corresponding to different measurement site areas are configured with individual pressure exertion apparatuses such that the sensor units in the individual measurement site areas are mechanically decoupled from one another and independently adjustable.

3. The adjustable sensor support structure of claim 1, wherein the flexible bridge structure comprises:
two bendable wings comprising a first wing and a second wing, wherein one end of the first wing is attached to one side of the band, and one end of the second wing is attached to an opposite side of the band,
wherein open ends of each of the wings are folded back on one another to form an oval bridge, where the open ends of both of the first and second wings are free hanging over the oval bridge; and
wherein at least one of the sensor units is attached to the open end of at least one of the wings.

4. The adjustable sensor support structure of claim 1, wherein assembly of the flexible bridge structure comprises:
printing and/or etching circuitry onto a plastic film that may form at least part of the band, and laser cutting the plastic film to form a shape of two-stage foldable wings, wherein each of the wings is formed with a slit;
during a first folding stage, folding the wings away from the band such that the wings stand approximately perpendicular to the band;
during a second folding stage, folding an open end of the wings towards the band, causing the open end of the wings and material formed by the slit to lie approximately parallel to the band;
inserting the open ends of each of the wings into the slit in the opposite wing so that the wings align on top of each other and such that the two wings align, thereby forming a bridge that bends and flexes under pressure; and
affixing sensor units to the wings.

5. The adjustable sensor support structure of claim 1, further comprising multiple flexible bridge structures connected edge-to-edge on the band in a serial configuration.

6. The adjustable sensor support structure of claim 1, further comprising: multiple flexible bridge structures layered on top of each other to form a multiple bridge structure spring.

7. The adjustable sensor support structure of claim 1, wherein the flexible foam structure comprises:
a plurality of foam islands mounted upon the band, wherein at least a portion of each of the foam islands support at least one sensor unit;
isolation gaps formed between at least portions of the foam islands that are created from a shape of foam islands to allow expansion of the foam islands upon application of force to the sensor units; and
wire leads inserted through the internal cavities connecting the sensor units to the band.

8. The adjustable sensor support structure of claim 1, wherein the flexible foam structure comprises:
flexible cavity structures formed in a desired topology on the band, where at least a portion of flexible cavity structures support at least one sensor unit;
internal cavities formed in the flexible cavity structures that enable expansion volume upon compression of the flexible cavity structures upon application of force to the sensor units; and
at least one of a wire lead and a spring inserted through the internal cavities connecting the sensor units to the band.

9. The adjustable sensor support structure of claim 1, wherein the flexible foam structure comprises:
a sensor trampoline structure comprising a multi-dimensional spring-like mesh that supports multiple sensor units, the sensor trampoline structure being substantially constructed from wire and exhibiting spring tension in multiple dimensions to provide elastic support to the sensor units, allowing the sensor units to move independently in z-height, while twisting from side-to-side upon application of force to the sensor units;

10. The adjustable sensor support structure of claim 1, further comprising an active adjustment mechanism, wherein responsive to the sensor units detecting a physiologic signal from the body, a quality of the physiologic signal is received by the active adjustment mechanism and used to actively optimize sensor unit contact with the body.

11. The adjustable sensor support structure of claim 10 wherein optimal sensor topology settings for a particular user/body part are saved and used to identify a wearer of the adjustable sensor support structure.

12. A method of providing an adjustable sensor support structure, comprising:
providing a sensor array comprising a plurality of sensor units arranged on a band such that the sensor array straddles or otherwise addresses a blood vessel when worn on a measurement site of a user; and
attaching a pressure exertion apparatus between the sensor units and the band that exerts outward pressure on the sensor units towards the measurement site causing the sensor units to maintain contact with skin of the user independent of motion activity of the band, thereby improving contact quality,
wherein the pressure exertion apparatus comprises at least one of:
a flexible bridge structure,
a flexible foam structure, and
a sensor trampoline structure.

13. The method of claim 12, wherein sensor units corresponding to different measurement site areas are configured with individual pressure exertion apparatuses such that the sensor units in the individual measurement site areas are mechanically decoupled from one another and independently adjustable.

14. The method of claim 12, wherein the flexible bridge structure comprises:
two bendable wings comprising a first wing and a second wing, wherein one end of the first wing is attached to one side of the band, and one end of the second wing is attached to an opposite side of the band,
wherein open ends of each of the wings are folded back on one another to form an oval bridge, where the open ends of both of the first and second wings are free hanging over the oval bridge; and
wherein at least one of the sensor units is attached to the open end of at least one of the wings.

15. The method of claim 12, wherein assembly of the flexible bridge structure comprises:
printing and/or etching circuitry onto a plastic film that may form at least part of the band, and laser cutting the plastic film to form a shape of two-stage foldable wings, wherein each of the wings is formed with a slit;
during a first folding stage, folding the wings away from the band such that the wings stand approximately perpendicular to the band;
during a second folding stage, folding an open end of the wings towards the band, causing the open end of the wings and material formed by the slit to lie approximately parallel to the band;
inserting the open ends of each of the wings into the slit in the opposite wing so that the wings align on top of each other, thereby forming a bridge that bends and flexes under pressure; and
affixing sensor units to the wings.

16. The method of claim 12, further comprising: connecting multiple flexible bridge structures edge-to-edge on the band in a serial configuration.

17. The method of claim 12, further comprising: laying multiple flexible bridge structures on top of each other to form a multiple bridge structure spring.

18. The method of claim 12, wherein the flexible foam structure comprises:
a plurality of foam islands mounted upon the band, wherein at least a portion of each of the foam islands support at least one sensor unit;
isolation gaps formed between at least portions of the foam islands that are created from a shape of foam islands to allow expansion of the foam islands upon application of force to the sensor units; and
wire leads inserted through the internal cavities connecting the sensor units to the band.

19. The method of claim 12, wherein the flexible foam structure comprises:
flexible cavity structures formed in a desired topology on the band, where at least a portion of flexible cavity structures support at least one sensor unit;
internal cavities formed in the flexible cavity structures that enable expansion volume upon compression of the flexible cavity structures upon application of force to the sensor units; and
at least one of a wire lead and a spring inserted through the internal cavities connecting the sensor units to the band.

20. The method of claim 12, wherein the flexible foam structure comprises:
a sensor trampoline structure comprising a multi-dimensional spring-like mesh that supports multiple sensor units, the sensor trampoline structure being substantially constructed from wire and exhibiting spring tension in multiple dimensions to provide elastic support to the sensor units, allowing the sensor units to move independently in z-height, while twisting from side-to-side upon application of force to the sensor units;

21. The method of claim 12, further comprising: providing the adjustable sensor support structure with an active adjustment mechanism, wherein responsive to the sensor units detecting a physiologic signal from the body, a quality of the physiologic signal is received by the active adjustment mechanism and used to actively optimize sensor unit contact with the body.

22. The method of claim 21, further comprising: saving optimal sensor topology settings for a particular user/body part and using the optimal sensor topology settings to identify a wearer of the adjustable sensor support structure.
